(19) United States
(12) Patent Application Publication
(10) Pub. No.: US 2006/0057107 A1
(43) Pub. Date: Mar. 16, 2006

(54) Combination treatment for multiple sclerosis

(76) Inventors: Ze'ev Shaked, San Antonio, TX (US); Richard P. Bryce, Boerne, TX (US); Sonny B. Fong, LaSalle (CA)

Correspondence Address: Steven L Highlander;Fulbright & Jaworski, 600 Congress Avenue, Suite 2400, Austin, TX, 78701, US

(21) Appl. No.: 10/499,493

(22) PCT Filed: Dec. 19, 2002
(86) PCT No.: PCT/US02/40851

§ 371 Date: Nov. 14, 2005

Related U.S. Application Data
(60) Provisional application No. 60/342,939, filed on Dec. 21, 2001.
(60) Provisional application No. 60/387,795, filed on Jun. 11, 2002.
(60) Provisional application No. 60/319,531, filed on Sep. 6, 2002.

(51) Int. Cl. A61K 38/21; A61K 39/395

(52) U.S. Cl. 424/85.7; 424/144.1

(57) ABSTRACT

The present invention provides for the treatment of multiple sclerosis by combination therapies of CD52 specific antibodies, such as CAMPATH-1H, with either a Type 1 interferon or coploymer-1.

### FIELD OF THE INVENTION

The present invention relates to the use of CD52 specific antibodies in the treatment of autoimmune diseases. More particularly it relates to the use of anti-CD52 antibodies in combination with other therapeutic agents in the treatment of multiple sclerosis.

### BACKGROUND OF THE INVENTION

Multiple sclerosis (“MS”) is a chronic and progressive inflammatory disease of the central nervous system (“CNS”). The disease is characterized by loss of the myelin layer that insulates nerve fibers and, while the precise cause of MS is unknown, it is generally recognized that there is an autoimmune component in the etiology of the disease. The areas of demyelination, or plaques, are inflammatory in nature with infiltration by T and B-lymphocytes and macrophages. There may also be a decrease in T-suppressor cell number and function (Ffrench-Constant, 1994). The degree of inflammation correlates with the amount of axonal loss. Chronic lesions lose their components in nature and are characterized by demyelinated axons separated by a dense network of astrocyte processes (Trapp et al., 1998).

Patients with multiple sclerosis can be generally categorized into different groups (Weinshenker, 1995). Relapsing remitting multiple sclerosis (“RR-MS”) is characterized by periodic exacerbations of MS symptoms followed by periods of remission with complete or near-complete recovery. In secondary progressive multiple sclerosis (“SP-MS”) there are distinct attacks as in RR-MS, but the intervening recovery is incomplete and there is a progression of disability in the “recovery” phases. In primary progressive multiple sclerosis (“PP-MS”), there is a rapid deterioration in condition to severe disability with no discernable periods of remission.

There is currently no cure for MS, although a number of drugs are used to treat the symptoms of the disease. Interferon-β is one of the most effective drugs available that can inhibit the progression of the disease (Smith & Darlington, 1999). Glatiramer acetate/copolymer I (Copaxone®) has also shown ability to inhibit disease progression in RR-MS (Johnson et al., 1998). Corticosteroids, particularly methylprednisolone, have been used extensively in the treatment of relapses and acute exacerbations (Bansil et al., 1995). Numerous other drugs have been used for specific aspects of symptomatic treatment, such as for fatigue and motor, urinary, sensory and psychological symptoms (Smith & Darlington, 1999).

While there are a number of treatment modalities for MS, there is a current need for improved treatments for this chronic disabling disease.

### SUMMARY OF THE INVENTION

The present invention provides for the treatment of MS by combination therapies comprising administration of CD52-specific antibodies with either a Type 1 interferon or copolymer-1, such combination therapies providing greater benefits than those associated with the corresponding single-agent treatment regimens. One aspect of the invention provides for a method for the treatment of an MS patient comprising administering an anti-CD52 antibody as a first treatment modality and Type 1 interferon as a second treatment modality, wherein the combined administration is effective to treat MS. The Type 1 interferon may be an interferon-α or interferon-β. In a preferred embodiment, the anti-CD52 antibody is CAMPATH-1H. In some embodiments, the interferon-β is an interferon-β-1b, which may be Rebif®D or Avonex® or the interferon-β is an interferon-β-1b, which may be Betaseron®. The interferon-α may be interferon-α2a which may be ROFERON®, or the interferon-α may be interferon-α2b which may be INTRON® A or PEG-INTRON™.

Another aspect of the invention provides for a method for the treatment of an MS patient comprising administering an anti-CD52 antibody as a first treatment modality and copolymer-1 as a second treatment modality, wherein the combined administration is effective to treat MS. In a preferred embodiment, the anti-CD52 antibody is CAMPATH-1H. In some embodiments, the copolymer-1 is Copaxone®.

### DETAILED DESCRIPTION OF THE INVENTION

A. CD52 Specific Antibodies

The CD52 (CAMPATH-1) antigen is a glycoprotein expressed on lymphocytes, monocytes, macrophages, NK cells, and tissues of the male reproductive system (Hale et al., 1990). Antibodies to CD52 are disclosed in U.S. Pat. No. 5,846,534, herein incorporated by reference. The use of CD52 specific antibodies for the treatment of MS is disclosed by U.S. Pat. No. 6,120,766, herein incorporated by reference. Anti-CD52 antibodies bind to all lymphocytes, a majority of monocytes, macrophages, and NK cells, and a subpopulation of granulocytes, but lyse only lymphocytes in vivo. CAMPATH-1M is a rat IgM monoclonal antibody that has been used extensively to deplete T-cells in bone marrow harvests prior to transplantation. CAMPATH-1G is a rat IgG2b class-switch variant of a IgG2a antibody. This antibody has been used in vivo for immunosuppression in transplant patients. CAMPATH-1H is a humanized monoclonal antibody and is approved for the treatment of B-cell chronic lymphocytic leukemia in patients who have been treated with alkylating agents and who have failed fludarabine therapy. CAMPATH-1H is distributed as CAMPATH® (Alemtuzumab) in the U.S. (Berlex) and MABCAMPATH™ in Europe (Schering A. G.).

Infusion of CAMPATH-1H results in the rapid fall of lymphocyte and monocyte counts over the first hour post-treatment and a prolonged lymphopenia that ensues for over 2 years. Administration of CAMPATH-1H to patients with SP-MS resulted in a sustained and marked decrease in active inflammation and the prevention of new symptoms, although some patients experienced progressive disability and increasing brain atrophy associated with axonal loss (Paolillo et al., 1999; Coles et al., 1999). Axonal degradation correlated with the extent of cerebral inflammation in the pretreatment phase.

B. Interferon-β

There are three recombinant interferon-β products available for the treatment of MS: Rebif® (Serono); Avonex® (Biogen); and Betaseton® (Bertex). The later is a mutant lacking the N-terminal methionine and serine substituted for cysteine at the 17 position. Betaseron® is made in Escherichia coli and lacks the glycosylation of the native molecule and is thereby classified as an interferon-β-1b. Rebif® and Avonex® have the native interferon-β sequence and being produced in CHO cells are glycosylated and are thereby termed as interferon-β-1a's. The term “interferon-(3” as used herein encompasses both interferon-β-1a and interferon-β-1b variants.

In RR-MS patients, interferon-β decreases both the development of lesions and the occurrence of new lesions (Simon et al., 1998; Stone et al., 1997; Calabresi et al., 1997) and also has been reported to improve cognitive function (Pliskin et al., 1996). In SP-MS, interferon-β has been shown to delay the sustained neurological deterioration characteristic of these patients (European Study Group on Interferon β-1b in Secondary Progressive MS, 1998).

Although the mode of action of interferon-β remains uncertain, one proposed mechanism is via inhibition of the immunological effects and synthesis of interferon-γ (Pantich & Bever, 1993; Corsini et al., 1997; Peitereit et al., 1997). However, type 1 interferons (interferons α and β) may directly upregulate T-cell interferon-γ production (Karp et al., 2001), a conclusion that is consistent with observations that both circulating neopterin levels and MHC-Class II molecule expression on circulating monocytes rise when patients are started on interferon-β (Chiang et al., 1993; Spears et al., 1987). Further, the number of circulating interferon-γ secreting cells are increased in the first two months of treatment with interferon-β (Arnason et al., 1997). An alternative mechanism for interferon-β activity is via upregulation of interleukin-10 and downregulation of interleukin-12, resulting in decreased presentation of antigens implicated in demyelination and loss of oligodendrocytes associated with MS (Karp et al., 2001). The action of interferon-β on MS via modulation of the interleukin-10/interleukin-12 axis may be compromised by the direct upregulation of interferon-γ production. Interleukin-10, the dominant endogenous inhibitor of interleukin-12, is produced by a wide variety of cells, including antigen presenting cells, astrocytes and microglia in addition to T-cells. Interleukin-12 is produced predominantly by antigen presenting cells and a subset of B cells and to a lesser extent by astrocytes and microgoia (Karp et al., 2001). Consequently, while the present invention is not bound by any one theory, the interferon-β-induced interferon-γ production may be negated by removal of the interferon-γ T-cells by administration of anti-CD52 antibody, while leaving the interleukin10/interleukin-12 axis functional and thereby potentiating the efficacy of interferon-β.

C. Interferon-α

Interferon-α products currently marketed include ROFERON®, a recombinant interferon-α2a marketed by Hoffman-La Roche, Nutley N.J., and INTRON® A, a recombinant interferon-α2b marketed by Schering Corp., Kenilworth N.J. Schering also markets PEG-INTRON™, an interferon-α2b conjugated with monomethoxy polyethylene glycol (“PEG”), called.

In RR-MS patients, interferon-α2a reduces exacerbation rate and MRI signs of disease activity (Durelli et al., 1994; Durelli et al., 1996; Myhr et al., 1999). The immunomodulatory effects of interferon-α are broadly similar to those described above for interferon-β (Weinstock-Guttman et al., 1995; Panitch & Bever, 1993; Durelli et al., 1994; Bongioanni et al., 1996; Piazzolla et al., 2000; Byrnes et al., 2001)

D. Copolymer-1

Copolymer-1 (glatiramer acetate), a synthetic peptide analogue of myelin basic protein (BP), is a standardized mixture of L-glutamic acid, L-lysine, L-alanine and L-tyrosine with a molar ratio of 0.14:0.34:0.43:0.1 and a molecular mass of 4.7-11.0 KDa Copolymer-1 has been demonstrated to have beneficial effects on MRI-defined brain lesions and reduces the relapse rate and accumulated disability of RR-MS patients (Johnson et al., 2000, Johnson et al., 1998; Mancardi et al., 1998). This agent has been approved for the treatment of RR-MS in the U.S. and is distributed under the name Copaxone® (Teva Pharmaceuticals). The composition and preparation of copolymer-1 are disclosed in U.S. Pat. Nos. 5,981,589 and 6,054,430, both herein incorporated by reference.

Copolymer-1 is cross-reactive with MBP and may inhibit the immune response in MS to 4 MBP, proteolipid protein and/or myelin oilgodendrocyte glycoprotein (Arnon et al., 1996; Ben-Nun et al., 1996; Lea & Goa, 1996). A shift from a Th1-biased T-cell cytokine profile (II-2, IFN-γ, TNF-α) towards a Th2-biased cytokine profile (IL-4, IL-5, IL-6, IL-10, TFG-β) has been observed in copolymer-1 treated MS patients (Duda et al., 2000; Neuhaus et al., 2000). The copolymer-1 specific Th2-type T-cells may be involved in the copolymer-1-induced therapeutic effects in MS (Aharoni et al., 2000).

D. Formulations and Administration

The pharmaceutical compositions according to the present invention are prepared conventionally, comprising substances that are customarily used in pharmaceuticals, e.g., Remington's Pharmaceutical Sciences, 18th ed., Mack Publishing Company (1990), including excipients, carriers, adjuvants, and buffers. The compositions can be administered, e.g., parenterally, enterally, orally, intramuscularly, subcutaneously, intravenously, by aerosol, intrathecally directly into the cerebral spinal fluid of the CNS, or other routes useful to achieve an effect. For example: anti-CD52 antibodies, preferably CAMPATH-1H, can be given intravenously (Cloes et al., 1999; Moreau et al., 1996; Moreau et al., 1994, all herein incorporated by reference) and subcutaneously (Schnitzer et al., 1997; Bowen et al., 1997, both herein incorporated by reference); interferon-β may be given subcutaneously (Stone et al., 1997, herein incorporated by reference) and by intramuscular administration (Simon et al., 1998, herein incorporated by reference); interferon-α may be given subcutaneously (Durelli et al., 1994, herein incorporated by reference) and by intramuscular administration (Myhr et al., 1999, herein incorporated by reference); and copolymer-1 can be administered subcutaneously (Johnson et al., 2000, herein incorporated by reference) and intramuscularly (Jacobs et al., 1994, herein incorporated by reference).

Conventional excipients include pharmaceutically acceptable organic or inorganic carrier substances suitable for parenteral, enteral, or topical application that do not deleteriously react with the agents. Suitable pharmaceutically acceptable adjuvants include, but are not limited to water, salt solutions, alcohols, gum arabic, vegetable oils, polyethylene glycols, gelatine, lactose, amylose, magnesium stearate, talc, silicic acid, viscous paraffin, perfume oil, fatty acid monoglycerides and diglycerides, pentaerythritol fatty acid esters, hydroxy-methylcellulose, polyvinyl pyrrolidone, cyclodextrins, etc. The pharmaceutical preparations can be sterilized and, if desired, mixed with stabilizers, wetting agents, emulsifiers, salts for influencing osmotic pressure, buffers, coloring, flavoring and/or aromatic substances, etc., that do not react deleteriously with the active compounds.

For parenteral application, particularly suitable are injectable sterile solutions, preferably oil or aqueous solutions, as well as suspensions, emulsions or implants, including suppositories. Ampules are convenient unit dosages.

The compositions can also be formulated in an aqueous solution, optionally with the addition of additives customary in galenicals, for example, buffers; electrolytes such as sodium chloride; antioxidants such as ascorbic acid; adjuvants, e.g., methylcellulose, lactose and mannitol and/or surfactants, e.g., lecithins and Tweens and/or aromatic substances for flavoring, e.g., ethereal oils.

Dosage levels and treatment regimens for MS of interferon-β, including Rebif®, Avonex®, and Betaseron®, interferon-α, including ROFERON®, INTRON® A and PEG-INTRON™, and copolymer-1 preparations, including Copaxone®, are known in the art. E.g., the package insert instructions for Rebif indicate a subcutaneous dose of 44 mcg 3 times per week. A clinical study may suitably use an initial titration e.g., in the first 6 weeks Rebif is administered subcutaneously at 11 mcg 3 times per week for the first two weeks, 22 mcg 3 times a week for the next two weeks, and 33 mcg for the following two weeks. A patient that has an adverse reaction to Rebif may suitably have dose adjustments made in accordance to the physician's discretion in accordance with the clinical guidelines provided by the manufacturer. ROFERON may suitably be given in the range of about 4.5 mIU to about 9 mIU subcutaneoulsy or intramuscularly three times per week. Copoaxone may suitably be administered subcutaneously at a daily dose of 20 mg.

In some embodiments, the dosage of Type 1 interferons, i.e., interferon-α or interferon-β, when used in a combination regiment with an anti-CD52 antibody is reduced compared to the interferon-β dosage used in a single-agent treatment regimen. The dosage of a course of anti-CD52 antibodies, preferably CAMPATH-1H, may vary with the status of the MS patient and will generally be in the range of about 10 to about 150 mg for an adult patient, usually administered over a period from 1 to about 20 days. The course of treatment may be given once or may be repeated at about 3 month, or about six month, or at about 9 month, or about 12 month, or about 18 month or at about 24 month intervals, the number of courses of treatment depending upon the medical status of the patient, including but not limited, to the symptoms of MS and extent and persistence of lymphopenia. In some embodiments of the present invention, the dosage schedules suitably utilized in a clinical study are a low dose level of a total of 0.37 mg/kg, a mid dose level of a total of 0.75 mg/kg and a high dose level of a total of 1.50 mg/kg, all given IV over a total of 5 consecutive, i.e., 0.07, 0.15 and 0.30 mg/kg/day respectively. Re-treatment is given at months 24 and 48 months at a low dose level of a total of 0.22 mg/kg, a mid 4 dose level of a total of 0.45 mg/kg and a high dose level of a total of 0.90 mg/kg, all given IV over a total of 3 consecutive, i.e., 0.07, 0.15 and 0.30 mg/kg/day respectively.

The first course of CAMPATH-1H treatment has been associated with a reversible exacerbation of existing neurological symptoms and activation of asymptomatic lesions caused by an antibody-induced release of cytokines (Moreau et al., 1996; Wing et al., 1996). This cytokine-release syndrome can be prevented by pretreatment with methylprednisolone (Coles et al., 1999, herein incorporated by reference).

In accordance with the methods of the present invention, the two treatment modalities in the combination of anti-CD52 antibodies with interferon-α, interferon-β or copolymer-1 can be administered separately at different times during the course of therapy or concurrently in divided or single combination forms. In a preferred embodiment, the administration of an anti-CD52 antibody precedes the administration of interferon-β interferon-α. According to the instant invention, the term administering is to be understood as embracing all such regimes of simultaneous or alternating treatment and the scope of combinations of anti-CD52 antibodies with interferon-α, interferon-β or copolymer-1 includes, in principle, any combination useful for treating MS.

### EXAMPLES OF THE INVENTION

A. Clinical Evaluation of Combined Therapies

Patients will be evaluated for clinically definite MS (Poser et al., 1983, herein incorporated by reference). SP-MS patients will have a Kurtzke expanded disability status score (EDSS; Kurtzke, 1983, herein incorporated by reference) of between 3.0-7.0 inclusive, with a recorded history of a 1.0 point or more increase in the previous 2 years. Immunosuppressive or immunomodulatory treatment or other putative treatments for MS are not permitted for a defined period prior to entry into the trial. Suitable eligibility criteria are provided by Polman et al., 1995, herein incorporated by reference. RR-MS patients will have had at least two relapses within the last two years and to be free from steroid use for at least two months. Suitable treatment cohorts for CAMPATH-1H and interferon-β combined therapy include: (1) treatment with CAMPATH-1H; (2) treatment with interferon-β; and (3) treatment with CAMPATH-1H and interferon-β. Suitable treatment cohorts for CAMPATH-1H and interferon-α combined therapy include: (1) treatment with CAMPATH-1H; (2) treatment with interferon-α; and (3) treatment with CAMPATH-1H and interferon-α. Suitable treatment cohorts for CAMPATH-1H and copolymer-1 combined therapy include: (1) treatment with CAMPATH-1H; (2) treatment with copolymer-1; and (3) treatment with CAMPATH-1H and copolymer-1.

Efficacy of treatments are suitably monitored by MRI, wherein MRI studies are calculated at baseline and followed up periodically over a period of up to 5 years. MRI can obtain images that are proton density (PD) weighted, T1-weighted, and T2 weighted (Paty, 1993; Francis et al., 1995, both herein incorporated by reference). Suitable MRI measurement techniques/parameters include: brain volume; gadolinium enhancement for the evaluation of disruption of the blood-brain barrier and inflammation; evaluation of new T2 lesions as an indicator of inflammation; enlarging T2 lesions as an indicator of increasing inflammation; and T1 “black holes” for permanent demyelination and axonal loss (Adams et al., 1999; Coles et al., 1999; Simon et al., 1998; Bruck et al., 1997; Katz et al., 1993; Hawkins et al., 1991, all herein incorporated by reference). MS-related disability is evaluated over the same period according to the Kurtzke EDSS system. One efficacy endpoint is the proportion of patients without sustained accumulation of disability (“SAD”) at defined time points after initiation of treatment. SAD is defined as an increase of ≧1.0 point of the EDSS sustained over a six-month consecutive period. Other criteria of efficacy include number of relapses, time to first relapse and rate of cerebral atrophy.

B. Preclinical Evaluation of Combined Therapies

1. Mouse Model

Preclinical evaluation is performed in an experimental autoimmune encephalomyelitis (“EAE”) model, suitable antigens being myelin oligodendrocyte glycoprotein (“MOG”), myelin basic protein (“MBP”) and proteolipid protein (“PLP”). Female mice (e.g., 6 to 16 weeks of age) of a strain susceptible to EAE induction are used e.g., SLJ/J (u et al., 2001), (SWR×SJL/J)F₁ (Yu et al., 1996, Sobel et al., 1991), (SJL/J×BALB/c)F₁ (Aharoni et al., 2000), C57BL/6 (Zhang et al., 2002) or PL/J (Soos et al., 2002). Immunization is suitably carried out as detailed in the art (e.g., Du et al., 2001; Yu et al., 1996; Sobel et al., 1991; Aharoni et al., 2000; Zhang et al., 2002; Soos et al., 2002 and Gold et al., 2000, all herein incorporated by reference). Effective doses of interferon-α, interferon-β and copolymer-1 have been described in mouse models (Brod et al., 1995; Yu et al., 1996; Aharoni et al., 2000, all herein incorporated by reference).

Anti-mouse CD52 is given subcutaneously for a number of consecutive days (e.g., 5 days at days 8-13). Effective dose range is established by monitoring the depletion of CD52 positive cells, e.g., T-cells. The B7 antigen (B7-Ag) is the mouse homolog to CD52 (Tone et al., 1999), and a rat anti-mouse B7-Ag IgG2a monoclonal antibody is described by Kubota et al. (1990). Preferably, an IgG2b class-switch variant is isolated, e.g., by sib selection using red cell-linked antibodies to identify the desired secreted Ig by reverse passive haemagglutination (Hale et al., 1987, herein incorporated by reference).

Cohorts include control (vehicle only) and single agents and combined treatments of anti-mouse CD52 and either interferon-β or copolymer 1.

Mice are graded for clinical signs of EAE according to a suitable guideline, e.g., Grade 1—tail weakness or tail paralysis; Grade 2—hind leg paraparesis or hemiparesis; Grade 3—hind leg paralysis or hemiparalysis; Grade 4—complete paralysis (tetraplegy), moribund state, or death. Ataxia will be routinely assessed. A disease remission is defined as an improvement in disease score from either 3 or 4 to 1, or from 2, 3 or 4 to 0, that is maintained for at least 2 consecutive days. A relapse is defined as an increase in the clinical deficit of at least two points that lasted for at least 2 days. Body weight is measured pretest and daily through the test.

Clinical pathology comprises lymphocyte proliferative responses are suitably assessed in viable spleen cells, inguinal lymph node cells or peripheral blood mononuclear cells in mice sacrificed at suitable time points, e.g., days 15 and 60.

Histopathology comprises evaluation of brain and spinal cord from all rats. Inflammatory index is determined from the number of perivascular inflammatory infiltrates of each animal on an average of 15 complete cross sections of spinal cord. The degree of myelination is suitably evaluated separately for brain and spinal cord and scored, e.g., 0.5 traces of perivascular or subpial demyelination; 1=marked perivascular or subpvial demyelination; 2=confluent perivascular or subpial demyelination; 3=massive confluent demyelination (e.g., half of spinal cord, one complete optic nerve); and 4=extensive demyelination (transverse myelitis, half of the cerebellar white matter or more, both complete optic nerves).

2. Rat Model

Preclinical evaluation is again performed in an experimental autoimmune encephalomyelitis (“EAE”) model, suitable antigens being myelin oligodendrocyte glycoprotein (“MOG”), myelin basic protein (“MBP”) and proteolipid protein (“PLP”). Female rats (10 to 14 weeks of age) of a strain susceptible to EAE induction are used (e.g., DA, Lewis.1A, Lewis.AV1 or Lewis.1N-fulminant disease model). Rats are immunized utilizing a suitable regimen, e.g., intradermal injection at the base of the tail, e.g., 1, 5, 20, 50 or 100 μL, of an MOG inoculum, e.g., 1:1 MOG and saline emulsified with CFA (Sigma Chemical Co, ST Louis Mo.) containing 200 μg of Mycobacterium tuberculosis (strain H 37 RA, Difco Labs Irvine Calif.).

Anti-rat antibodies are raised against the rat B7-antigen homolog to CD52 (Kirchhoff, 1994, Eccleston et al., 1994), by methods generally know in the art. Anti-rat CD52 antibody is given subcutaneously for a number of consecutive days (e.g., 5 days at days 8-13 post-inoculation). Interferon-β is given by subcutaneous dose at a suitable starting point, e.g., day 8, and then every other day thereafter. Copolymer-1 is given by subcutaneous dose on a suitable starting point, e.g., day 8, and then daily thereafter. Cohorts include control (vehicle only) and single agents and combined treatments of anti-rat CD52 and either interferon-βor copolymer-1.

Rats are graded for clinical signs of EAE according to a suitable guideline, e.g.: Grade 1—tail weakness or tail paralysis; Grade 2—hind leg paraparesis or hemiparesis; Grade 3—hind leg paralysis or hemiparalysis; Grade 4—complete paralysis (tetraplegy), moribund state, or death. Ataxia will be routinely assessed. A disease remission is defined as an improvement in disease score from either 3 or 4 to 1, or from 2, 3 or 4 to 0, that is maintained for at least 2 consecutive days. A relapse is defined as an increase in the clinical deficit of at least two points that lasted for at least 2 days. Body weight is measured pretest and daily through the test.

Clinical pathology comprises lymphocyte proliferative responses are suitably assessed in viable spleen cells, inguinal lymph node cells or peripheral blood mononuclear cells in rats sacrificed at suitable timepoints, e.g., days 15 and 60.

Histopathology suitably comprises evaluation of brain and spinal cord from all rats. Inflammatory index is determined from the number of perivascular inflammatory infiltrates of each animal on an average of 15 complete cross sections of spinal cord. The degree of myelination will be evaluated separately for brain and spinal cord and scored, e.g.: 0.5=traces of perivascular or subpial demyelination; 1=marked perivascular or subpvial demyelination; 2=confluent perivascular or subpial demyelination; 3=massive confluent demyelination (e.g., half of spinal cord, one complete optic nerve); and 4 extensive demyelination (transverse myelitis, half of the cerebellar white matter or more, both complete optic nerves).

The present invention has been shown by both description and examples. The examples are only for exemplification and cannot be construed to limit the scope of the invention. One of ordinary skill in the art will envision equivalents to the inventive process described by the following claims that are within the scope and spirit of the claimed invention.

### REFERENCES

The following references, to the extent that they provide exemplary procedural or other details supplementary to those set forth herein, are specifically incorporated herein by reference.
Adams et al., “Hypointense and hyperintense lesions on magnetic resonance imaging in secondary-progressive MS patients,” Eur. Nurol., 42:52-63, 1999.
Arnon et al., “New insights into the mechanism of action of copolymer-1 in experimental allergic encephalomyelitis and multiple sclerosis,” J. Neurol., 243(Suppl. 1):S8-S13, 1996.
Aharoni et al., “Specific Th2 cells accumulate in the central nervous system of mice protected against experimental autoimmune encephalomyelitis by copolymer 1,” Proc. Natl. Acad. Sci. USA, 97:11472-77, 2000.
Arnason et al., “Role of interferons in demyelinating diseases,” J. Neural. Transm., 49:117-23, 1997.
Bansil et al., “Multiple sclerosis: immune mechanisms and update on current therapies,” Ann. Neurol., 37(S1):S87-S101, 1995.
Ben-Nun et al., “The autoimmune reactivity to myelin oilgodendrocyte glycoprotein (MOG) in multiple sclerosis is potentially pathogenic—effect of copolymer-1 on MOG-induced disease,” J. Neurol., 243(Suppl. 1):S14-S22, 1996.
Bongioanni et al., “Systemic high-dose recombinant-alpha-2a-interferon therapy modulates lymphokine production in multiple sclerosis,” J. Neurol. Sci., 143:91-99, 1996.
Bowen et al., “Subcutaneous CAMPATH-1H in fludarabine-resistant/relapsed chronic lymphocytic and B-prolymphocytic leukemia,” Br. J. Hematol., 96:617-9, 1997.
Brod et al., “Oral administration of human or murine interferon alpha suppresses relapses and modifies adoptive transfer in experimental autoimmune encephalomyelitis, J. Neuroimmunol, 58:61-69, 1995.
Bruck et al., “Inflammatory central nervous system demyelination: correlation of magnetic resonance imaging findings with lesion pathology,” Ann. Neurol., 42:783-93, 1997.
Byrnes et al., “Type 1 interferons and IL-12: convergence and cross-regulation among mediators of cellular immunity,” Eur. J. Immunol., 31:2026-2034, 2001.
Calabresi et al., Interferon beta results in immediate reduction of contrast-enhanced MRI lesions in multiple sclerosis patients followed by weekly MRI.” Neurol., 48:1446-48, 1997.
Chiang et al., “Pharmacokinetics of recombinant human interferon-bₛₑᵣ in healthy volunteers and its effects on serum neopterin,” Pharm. Res., 10:567-72, 1993.
Coles et al., “Monoclonal antibody treatment exposes three mechanisms underlying the clinical course of multiple sclerosis,” Ann. Neurol., 46:296-304, 1999.
Caorsini et al., “Effects of beta-interferon-1b treatment in MS patients on adhesion between PBMNCs, HUVECs and MS-HBECs—an in vivo and in vitro study,” J. Neuroimmunol., 79:76-83, 1997
European Study Group on Interferon β-1b in Secondary Progressive MS, “Placebo-controlled multicentre randomized trial of interferon β-1b in treatment of secondary progressive multiple sclerosis,” Lancet, 352:1491-97, 1998.
Duda et al., “Glatiramer acetate (Copaxone) induces degenerate, Th2-polarized immune responses in patients with multiple sclerosis” J. Clin. Invest., 105:967-76, 2000.
Du et al., “Administration of dehydroepiandrosterone suppresses experimental allergic encephalomyelitis in SJL/J mice,” J. Immunol., 167:1094-7101, 2001.
Durelli et al., “Chronic systemic high-dose recombinant interferon alfa-2a reduces exacerbation rate, MRI signs of disease activity, and lymphocyte interferon gamma production in relapsing-remitting multiple sclerosis,” Neurol., 44:406-413, 1994.
Durelli et al., “Interferon alfa-2a treatment of relapsing-remitting multiple sclerosis,” Neurol., 47:123-129, 1996.
Eccleston et al., “Characterization of a cell surface glycoprotein associated with maturation of rat spermatozoa,” Mol. Reprod. dev., 37:110-119, 1994.
Ffrench-Constant, Pathogenesis of multiple sclerosis,” Lancet, 343:271-5, 1994.
Francis et al., “Neuroimaging in multiple sclerosis,” Neurol. Clin., 13:147-71, 1995.
Gold et al., “Animal models for autoimmune demyelinating disorders of the nervous system,” Mol. Med. Today, 6:88-91, 2000.
Hale et al., “The CAMPATH-1 antigen (CDw52), Tissue Antigens,” 35:118-27, 1990.
Hale et al., “Isolation of low-frequency class-switch variants from rat hybrid myelomas,” J. Immunol. Methods, 103:59-67, 1987.
Hawkins et al., “Patterns of blood-brain barrier breakdown in inflammatory demyelination,” Brain, 114:801-10, 1991.
Johnson et al., “Extended use of glatiramer acetate (Copaxone®) is well tolerated and maintains its clinical effect on multiple sclerosis relapse rate and degree of disability,” Neurol, 50:701-8, 1998.
Jacobs et al., “Advances in specific therapy for multiple sclerosis,” Curr. Opin. Neurol., 7:250-4, 1994.
Johnson et al., “Sustained clinical benefits of glatiramer acetate in relapsing multiple sclerosis observed for 6 years. Copolymer 1 Multiple Sclerosis Group,” Multiple Sclerosis, 6:255-66, 2000.
Karp et al., “Interferon-β in multiple sclerosis: altering the balance of interleukin-12 and interleukin-10?” Curr. Opin. Neurol., 14:361-68, 2001.
Katz et al., “Correlation between magnetic resonance imaging findings and lesion development in chronic, active multiple sclerosis,” Ann. Neurol., 34:661-69, 1993.
Kirchhoff, “A major messenger ribonucleic acid of the rodent epidiymis encodes a small glycosylphosphatidylinositol-anchored lymphocyte surface antigen,” Biol. Rep., 50:896-902, 1994.
Kurtzke, “Rating neurological impairment in multiple sclerosis: an expanded disability scale (EDSS),” Neurol., 33:1444-52, 1983.
Lea & Goa., “Coploymer-1—A review of its pharmacological properties and therapeutic potential in multiple sclerosis,” Clin. Immunotherap., 6:319-31, 1996.
Mancardi et al., “Effect of copolymer-1 on serial gadloinium-enhanced MRI in relapsing remitting multiple sclerosis,” Neurol., 50:1127-33, 1998.
Moreau et al., “Preliminary evidence from magnetic resonance imaging for reduction in disease activity after lymphocyte depletion in multiple sclerosis,” Lancet, 344:298-301, 1994.
Moreau et al., “Transient increase in symptoms associated with cytokine release in patients with multiple sclerosis,” Brain, 119:225-37, 1996.
Moreau et al., “CAMPTH-IH in multiple sclerosis,” Multiple Sclerosis, 1:357-65, 1996.
Myhr et al., “Interferon-α2a reduces MRI disease activity in relapsing-remitting multiple sclerosis,” Neruol., 52:1049-'056, 1999.
Neuhaus et al., “Multiple sclerosis: comparison of copolymer-1—reactive T cell lines from treated and untreated subjects reveals cytokine shift from T helper 1 to T helper 2 cells,” Proc. Natl. Acad. Sci. USA, 97:7452-57, 2000.
Panitch & Bever, “Clinical trials of interferons in multiple sclerosis. What have we learned?” J. Neuroimmunol., 46:155-64, 1993.
Paolillo et al., “Quantitative MRI in patients with secondary progressive MS treated with monoclonal antibody Campath 1H,” Neurol., 53:751-7, 1999.
Paty, “Magnetic resonance in multiple sclerosis,” Curr. Opin. Neurol. Neurosurg., 6:202-8, 1993.
Peitereit et al., “Interferon gamma producing blood lymphocytes are decreased by interferon beta therapy in patients with multiple sclerosis,” Multiple Sclerosis, 3:180-83, 1997.
Piazzolla et al., “Relationship between interferon-gamma, interleukin-10, and interleukin-12 production in chronic hepatitis C and in vitro effects of interferon-alpha,” J. Clin. Immunol., 20:54-61, 2000.
Pliskin et al., “Improved delayed visual reproduction test performance in multiple sclerosis patients receiving interferon β-b,” Neurol., 47:1463-68, 1996.
Polman et al., “Interferon beta-1b in secondary-progressive multiple sclerosis: outline of the clinical trial,” Multiple Sclerosis, 1(Suppl.)51-54, 1995
Poser et al., “New diagnostic criteria for multiple sclerosis: guidelines for research protocols,” Ann. Neurol., 13:227-31, 1983.
Schnitzer et al., “Subcutaneous administration of CAMPATH-1H: clinical and biological outcomes,” J. Rheumatol., 24:1031-6, 1997.
Simon et al., “Magnetic resonance studies of intramuscular interferon β-a for relapsing multiple sclerosis,” Ann. Neurol., 43:79-87, 1998.
Smith & Darlington, “Recent developments in drug therapy for multiple sclerosis,” Multiple Sclerosis, 5:110-20, 1999.
Sobel et al., “Parental MHC molecule haplotype expression in (SJL/J×SWR) F₁ mice with acute experimental allergic encephalomyelitis induced with different synthetic peptides of myelin proteolipid protein,” J. Immunol., 146:543-549, 1991.
Soos et al., “Intramolecular epitope spreading induced by staphylococcal enterotoxin superantigen reactivation of experimental allergic encephalomyelitis,” J. Neuroimmunol., 123:30-34, 2002.
Spear et al., “Enhancement of monocyte class I and II histocompatibility antigen expression in man by in vivo interferon,” Clin. Exp. Immunol., 69:107-15, 1987.
Stone et al., “Characterization of MRI response to treatment with interferon beta-1 b,” Neurol., 49:862-69, 1997.
Tone et al., “Structural and chromosomal location of mouse and human CD52 genes,” Biochim. Biophys. Acta, 1446:334-340, 1999.
Trapp et al., “Axonal transection in the lesions of multiple sclerosis,” N. Engl. J. Med., 338:278-85, 1998.
Weinshenker, “The natural history of multiple sclerosis,” Neurol. Clin., 13:119-46, 1995.
Weinstock-Guttman et al., “The interferons: Biological effects, mechanisms of action, and use in multiple sclerosis,” Ann. Neurol., 37:7-15, 1995
Wing et al., “Mechanism of first-dose cytokine-release syndrome by CAMPATH 1-H: involvement of CD16 (FcγRIII) and CD11a/CD18 (LFA-1) on NK cells,” J. Clin. Invest., 12:2819-26, 1996.
Yu et al., Interferon-β inhibits progression of relapsing-remitting experimental autoimmune encephalomyelitis,” J. Neuroimmunol, 64:91-100, 1996.
Zhang et al., “Parenchymal microganglia of naive adult C57BL/6J mice express high levels of B7.1, B7.2 MHC class II,” Exp. Mol. Pathol., 73, 35-45, 2002.

1. A method for the treatment of a human subject suffering from multiple sclerosis, comprising administering to said subject an anti-CD52 antibody as a first treatment modality and a Type 1 interferon as a second treatment modality, wherein the administration of said anti-CD52 Yb antibody when combined with the administration of said Type 1 interferon-β is effective to treat said multiple sclerosis.

2. The method of claim 1, wherein said anti-CD52 antibody is CAMPATH-1H.

3. The method of claim 1, wherein said Type 1 interferon is interferon-β.

4. The method of claim 3, wherein said interferon-β is interferon-β-1a or interferon-β-1b.

5. The method of claim 4, wherein said interferon-β-1a is Rebif® or Avonex®.

6. The method of claim 4, wherein said interferon-β-1b is Betaseron®.

7. The method of claim 3, wherein said Type 1 interferon is interferon-α.

8. The method of claim 7, wherein said interferon-α is interferon-α2a or interferon-α2b.

9. The method of claim 8, wherein said interferon-α2a is ROFERON®.

10. The method of claim 8, wherein said interferon-α2b is INTRON® A or PEG-INTRON™.

11. A method for the treatment of a human subject suffering from multiple sclerosis, comprising administering to said subject an anti-CD52 antibody as a first treatment modality and copolymer-1 as a second treatment modality, wherein the administration of said anti-CD52 antibody when combined with the administration of said copolymer-1 is effective to treat said multiple sclerosis.

12. The method of claim 11, wherein said anti-CD52 antibody is CAMPATH-1H.

13. The method of claim 12, wherein said copolymer-1 is Copaxone®.
